(19) United States
(12) Patent Application Publication
(10) Pub. No.: US 2019/0365631 A1
(43) Pub. Date: Dec. 5, 2019

(54) NAIL ENAMEL COMPOSITION

(71) Applicant: L'ORÉAL, Paris (FR)

(72) Inventors: Jianxin FENG, Clark, NJ (US); Ramakrishnan HARIHARAN, Springfield, NJ (US)

(73) Assignee: L'ORÉAL, Paris (FR)

(21) Appl. No.: 15/992,370

(22) Filed: May 30, 2018

(51) Int. Cl. A61K 8/81; A61Q 3/02; A61K 8/37; A61K 8/34; A61K 8/73

(52) U.S. Cl. CPC A61K 8/8141; A61Q 3/02; A61K 8/731; A61K 8/34; A61K 8/37

(57) ABSTRACT

The invention presents a fast-drying nail enamel composition with good stability, easy application, high shine and long wear. The disclosed combination of an acrylic resin having a T_{g} greater than a threshold temperature; a second resin having a T_{g} less than the threshold temperature; at least one plasticizer selected from the group consisting of a benzoic acid derivative, a citric acid derivative, and an isobutyrate derivative; a non-polar silicone copolymer; and at least one solvent having an evaporation rate greater than butyl acetate provides a nail polish that can be dried within 40-60 seconds on fingernails without sacrificing other critical properties such as shine and wear resistance.

### FIELD OF THE INVENTION

The present invention relates to nail enamel compositions, and specifically to fast-drying nail enamel compositions comprising two different resins.

### BACKGROUND

Consumers use nail enamel to cosmetically enhance the appearance of their nails or protect the nails from the abuses found in their everyday environment. Often this requires a nail enamel that is formulated to provide a good shine.

Consumers also desire a durable nail enamel. Lack of durability is often evidenced by unsightly chipping or peeling of the coating soon after the original coating has been applied, requiring at least in part a reapplication of the coating in an attempt to recreate the aesthetic appearance or the therapeutic benefits of the original nail coating. Application and/or removal of more durable nail coatings is often very time consuming, requiring long cure times upon administration by skilled personnel leading to added costs for the consumer.

Modern, fast-paced consumers also desire a nail enamel with an easy application that dries rapidly. In many cases, fast-drying speed can be only achieved through sacrificing high shine, long wear, or storage stability. A fast-drying nail enamel, that avoids UV curing to speed the drying time, is therefore highly desirable.

### BRIEF SUMMARY

In a first aspect, the present invention is directed to a nail composition having (a) an acrylic resin having a T_{g} greater than a threshold temperature, (b) a second resin having a T_{g} less than the threshold temperature, (c) at least one plasticizer selected from the group consisting of a benzoic acid derivative, a citric acid derivative, and an isobutyrate derivative, (d) a non-polar silicone copolymer, and (e) at least one solvent having an evaporation rate greater than butyl acetate.

It is advantageous if that the threshold temperature is 75° C.

It is further advantageous when the acrylic resin is styrene/acrylates copolymer. The acrylic resin is present in an amount ranging from about 1% to about 10% by weight.

The second resin may advantageously be selected from the group consisting of a polyester, a polyurethane, acrylates copolymer, epoxy resin, alkyd resin, and a copolymer having trimellitic anhydride as a monomer, and in some instances, the copolymer having trimellitic anhydride as a monomer may be adipic acid/neopentyl glycol/trimellitic anhydride copolymer. Further, the second resin may be present in an amount ranging from about 1% to about 20% by weight.

Advantageously, the ratio of the acrylic resin to the second resin is between about 1:1 and about 1:5

The at least one plasticizer may include dipropylene glycol dibenzoate, acetyl tributyl citrate, and/or sucrose acetate isobutyrate. The plasticizer may be present in an amount ranging from about 1% to about 15% by weight.

The non-polar silicone copolymer may be acrylates/dimethicone copolymer, acrylates/stearyl acrylate/dimethicone acrylates copolymer, and/or acrylates/behenyl acrylate/dimethicone acrylates copolymer. Further, the non-polar silicone copolymer may be present in an amount ranging from about 0.01% to about 5% by weight.

The at least one solvent may advantageously be ethyl acetate, and in instances involving two or more solvents, the other solvents may include propyl acetate and isopropanol. The at least one solvent may present in an amount ranging from about 10% to about 70% by weight.

Advantageously, the nail composition may also include at least one pigment, and the pigment may be present in an amount greater than about 0.5% by weight. The nail composition may also nitrocellulose.

### DETAILED DESCRIPTION

As used herein, articles such as “a” and “an” when used in a claim, are understood to mean one or more of what is claimed or described.

As used herein, the term “about [a number]” is intended to include values rounded to the appropriate significant digit. Thus, “about 1” would be intended to include values between 0.5 and 1.5, whereas “about 1.0” would be intended to include values between 0.95 and 1.05.

As used herein, the term “at least one” means one or more and thus includes individual components as well as mixtures/combinations.

As used herein, the terms “include”, “includes” and “including” are meant to be non-limiting.

The present invention is directed to a nail composition having (a) an acrylic resin having a T_{g} greater than a threshold temperature, (b) a second resin having a T_{g} less than the threshold temperature, (c) at least one plasticizer selected from the group consisting of a benzoic acid derivative, a citric acid derivative, and an isobutyrate derivative, (d) a non-polar silicone copolymer, and (e) at least one solvent having an evaporation rate greater than butyl acetate. Each of these will be discussed in turn.

According to embodiments of the present invention, the nail composition includes an acrylic resin having a T_{g} greater than a threshold temperature. In preferred embodiments, the threshold temperature is between 60° C. and 90° C. In more preferred embodiments, the threshold temperature is between 70° C. and 80° C. In still more preferred embodiments, the threshold temperature is 75° C.

In certain embodiments, the acrylic resin can be a styrene/acrylate copolymer and, in particular, a polymer selected from copolymers obtained by polymerizing at least one styrene monomer and at least one (C₁-C₁₈) alkyl (meth)acrylate monomer. As a styrene monomer which can be used in the invention, mention may be made of styrene or alpha-methylstyrene, preferably styrene.

The (C₁-C₁₈) alkyl (meth)acrylate monomer is preferably a (C₁-C₁₂) alkyl (meth) acrylate and more preferably a (C₁-C₁₀) alkyl (meth)acrylate. The (C₁-C₁₈) alkyl (meth)acrylate monomer is selected from methyl acrylate, methyl methacrylate, ethyl acrylate, propyl acrylate, butyl acrylate, butyl methacrylate, hexyl acrylate, octyl acrylate, 2-ethylhexyl acrylate, lauryl (meth)acrylate and stearyl (meth)acrylate.

A suitable acrylic resin may include, but is not limited to, those sold under the Isocryl® name by Estron Chemical, including but not limited to Isocryl® C70. Further, the resin may include a polymer having an acid number of greater than 5, preferably between 5 and 150, more preferably between 20 and 100, and still more preferably between 65 and 75.

In certain embodiments, the acrylic resin is present in an amount ranging from about 1% to about 12% by weight. Preferably, the resin is present in an amount from about 1% to about 10%. More preferably, the resin is present in an amount from about 2% to about 8%. Still more preferably, the acrylic resin is present in an amount from about 4% to about 6%.

According to embodiments of the present invention, the nail composition also includes a second resin having a T_{g} less than the threshold temperature.

The second resin may preferably be a polyester, a polyurethane, an epoxy resin, an alkyd resin, an acrylates copolymer, and/or a copolymer having trimellitic anhydride as a monomer. In certain embodiments, the copolymer having trimellitic anhydride as a monomer is adipic acid/neopentyl glycol/trimellitic anhydride copolymer. Potential copolymers having trimellitic anhydride as a monomer include, but are not limited to, those sold commercially under the name Uniplex 670P.

In certain embodiments, the second resin may be a polymer comprising repeating units of at least one (meth)acrylate unit and vinyl copolymers. Potential acrylates copolymers include, but are not limited to, those sold under the PECOREZ® name such as, for example, PECOREZ AC 50.

In certain embodiments, the second resin may be a slightly branched, hydroxyl-bearing polyester. Potential slightly branched, hydroxyl-bearing polyesters for use in the present invention include, but are not limited to, those sold under the BAYCUSAN® name such as, for example, BAYCUSAN® XP 8000.

In certain embodiments, the second resin may be a polyurethane in solvents, having between about 20% and about 70% solids content. Certain embodiments may be aliphatic resins. Potential urethanes for use in the present invention include, but are not limited to, those sold under the REZE-LASTIC™ or REZE-MERIC™ name such as, for example, REZE-LASTIC™ 2140, REZE-MERIC™ 3182, or REZE-MERIC™ 3197.

In certain embodiments, the second resin may be an epoxy resin. Potential epoxy resins for use in the present invention include, but are not limited to, those sold under the POLYTEX™ name by Estron Chemical, Inc., such as, for example, POLYTEX™ NX-55.

In certain embodiments, the second resin may be an alkyd resin. Potential alkyd resins for use in the present invention include, but are not limited to, phthalic anhydride/glycerol/glycidyl decanoate copolymer, sold by Dainippon Ink & Chemicals.

The second resin is present in an amount ranging from about 1% to about 20% by weight, more preferably from about 1% to about 15% by weight, even more preferably from about 1% to about 10% by weight, and still more preferably from about 3% to about 8% by weight.

In certain embodiments, the weight ratio of the acrylic resin to the second resin is between about 1:0.5 and about 1:5, more preferably from about 1:0.8 to about 1:3, and still more preferably from about 1:0.9 to about 1:1.5.

According to embodiments of the present invention, the nail composition also includes at least one plasticizer selected from the group consisting of a benzoic acid derivative, a citric acid derivative, and an isobutyrate derivative.

In certain embodiments, the benzoic acid derivative may be a dibenzoate ester, including but not limited to diethylene glycol dibenzoate, dipropylene glycol dibenzoate, and 1,2-propylene glycol dibenzoate.

In certain embodiments, the citric acid derivative may be an optionally hydroxylated triester of a C₂-C₈ tricarboxylic acid and of a C₂-C₈ alcohol, such as citric acid esters, such as trioctyl citrate, triethyl citrate, acetyl tributyl citrate, tributyl citrate or acetyl tributyl citrate.

In certain embodiments, the isobutyrate derivative may be a C₁-C₆ carboxylic acid ester of sucrose such as sucrose acetate isobutyrate

In certain embodiments, the at least one plasticizer may be one or more of dipropylene glycol dibenzoate, acetyl tributyl citrate, and/or sucrose acetate isobutyrate.

The at least one plasticizer may be present in an amount ranging from about 1% to about 20% by weight, more preferably from about 1% to about 15% by weight, and still more preferably from about 4% to about 11% by weight.

According to embodiments of the present invention, the nail composition also includes a non-polar silicone copolymer. In certain embodiments, the non-polar silicone copolymer may comprise repeating units of at least one polar (meth)acrylate unit and vinyl copolymers grafted with at least one non-polar silicone chain. Non-limiting examples of such copolymers are acrylates/dimethicone copolymers such as those commercially available from Shin-Etsu, for example, the product sold under the tradename KP-543 KP-545, or acrylates/stearyl acrylate/dimethicone acrylates copolymers, such as those commercially available from Shin-Etsu, for example, the product sold under the tradename KP-561, and acrylates/behenyl acrylate/dimethicone acrylates copolymer, such as those commercially available from Shin-Etsu, for example, the product sold under the tradename KP-562.

The non-polar silicone copolymer may present in an amount ranging from about 0.01% to about 5% by weight. Preferably, the non-polar silicone copolymer may present in an amount ranging from about 0.01% to about 2% by weight, more preferably from about 0.01% to about 0.5% by weight, and still more preferably from about 0.05% to about 0.15% by weight.

According to embodiments of the present invention, the nail composition also includes at least one solvent having an evaporation rate greater than butyl acetate. In certain embodiments, the solvent comprises at least one acetic acid ester having a C₁-C₄ alkyl group, including but not limited to, methyl acetate, ethyl acetate, n-propyl acetate, and isopropyl acetate. For example, in some embodiments, the at least one solvent consists of ethyl acetate. In certain embodiments, the solvent comprises at least one alcohol having a C₁-C₄ alkyl group. For example, in some embodiments, the at least one solvent consists of ethyl acetate and at least one of either propyl acetate and isopropanol.

In certain embodiments, the solvent comprises a combination of at least one solvent having an evaporation rate greater than butyl acetate and at least one solvent having evaporation rate less than or equal to butyl acetate. For example, in some embodiments, the at least one solvent consists of ethyl acetate, propyl acetate, isopropanol, and butyl acetate.

The at least one solvent may be present in an amount ranging from about 1% to about 70% by weight. In preferred embodiments, the at least one solvent may be present in an amount ranging from about 5% to about 70% by weight, more preferably from about 10% to about 70% by weight, and more preferably from about 45% to about 55% by weight. In certain embodiments, the content of all evaporative solvents, including, e.g., butyl acetate, are present in an amount between about 60% and about 70% by weight.

According to embodiments of the present invention, the nail composition may also optionally include a variety of other components.

One of ordinary skill in the art would readily understand the types of ingredients typically found in nail compositions. A non-exhaustive list of such ingredients includes, but is not limited to, cellulose compounds and coloring agents.

Suitable cellulose compounds include, but are not limited to, cellulose polymers, such as, for example, hydroxyethylcellulose, hydroxypropylcellulose, methylcellulose, ethylhydroxyethylcellulose and nitrocellulose.

According to preferred embodiments, the at least one cellulose compound, if present, is present in the compositions of the present invention in an amount of active material ranging from about 0.01 to about 35% by weight, more preferably from about 2% to about 15% by weight, and most preferably from about 5% to about 15% by weight, based on the total weight of the composition, including all ranges and subranges within these ranges.

In certain embodiments, the nail composition also include at least colorant (coloring agent). Suitable colorants (coloring agents) include any colorant typically found in nail compositions. Suitable colorants include, but are not limited to, lipophilic dyes, pigments and pearlescent agents, and their mixtures. The at least one colorant, if present, may be present in an amount greater than 0.1% by weight, and typically less than about 5% by weight. Preferred embodiments utilize between about 0.5% and about 1% of colorant.

### Examples

The compositions listed in Table 1 (Ex. 1-3) were prepared by first premixing a 35% solution of styrene/acrylates copolymer in ethyl acetate. Then adding the remaining ingredients, excluding the pigments. The batches were then mixed until homogenous, then the pigments were added, and the mixing speed was increased until homogenous.

TABLE 1

| Material | Ex. 1 | Ex. 2 | Ex. 3 |
|---|---|---|---|
| Base film formers | 10.14 | 10.14 | 10.14 |
| Plasticizers (DBG, ATBC, SAIB) | 9.37 | 4.68 | 10.29 |
| Styrene/acrylates copolymers | 5.40 | 5.40 | 5.40 |
| Adipic Acid/Neopentyl Glycol/Trimellitic | 0.35 | 5.95 | 0.35 |
| Anhydride Copolymer | | | |
| Polyester | 5.4 | — | — |
| acrylates copolymer | .14 | .14 | 3.14 |
| Acrylates/Dimethicone Copolymer | 0.075 | 0.05 | 0.075 |
| Evaporative Solvents | 52.37 | 54.51 | 50.85 |
| Other Solvents (Butyl Acetate, etc.) | 12.72 | 15.10 | 15.72 |
| Colorants | 0.71 | 0.71 | 0.71 |
| Other Materials (Dimethicone, | 3.33 | 3.33 | 3.33 |
| Photoprotectors, etc.) | | | |

Evaluative testing results are shown in Table 2. Comparative examples without, e.g., the claimed plasticizer also dry rapidly, but chip after half a day.

TABLE 2

| | | | Touch | | |
|---|---|---|---|---|---|
| | | | Time To | Persoz | Persoz |
| | Shine at | Shine at | Dry | Hardness | Hardness |
| Formula | 20° (1 day) | 60° (1 day) | (seconds) | (1 day) | (7 day) |
| Ex. 1 | 65.9 +/− 0.8 | 87.8 +/− 0.1 | 50 | 56 +/− 5 | 61 +/− 5 |
| Ex. 2 | 53 +/− 0.3 | 83.1 +/− 0.1 | 40 | 58 +/− 2 | 49 +/− 1 |
| Ex. 3 | 54.4 +− 1.2 | 81.7 +/− 0.1 | 50 | 60 +/− 1 | 61 +/− 2 |

Those skilled in the art will recognize, or be able to ascertain using no more than routine experimentation, many equivalents to the specific embodiments of the invention described herein. Such equivalents are intended to be encompassed by the following claims.

What is claimed is:

1. A nail composition comprising:
an acrylic resin having a T_{g} greater than a threshold temperature;
a second resin having a T_{g} less than the threshold temperature;
at least one plasticizer selected from the group consisting of a benzoic acid derivative, a citric acid derivative, and an isobutyrate derivative;
a non-polar silicone copolymer; and
at least one solvent having an evaporation rate greater than butyl acetate.

2. The nail composition according to claim 1, wherein the threshold temperature is 75° C.

3. The nail composition according to claim 1, wherein the acrylic resin is present in an amount ranging from about 1% to about 10% by weight.

4. The nail composition according to claim 1, wherein the second resin is present in an amount ranging from about 1% to about 15% by weight.

5. The nail composition according to claim 1, wherein the ratio of the acrylic resin to the second resin is between about 1:1 and about 1:5.

6. The nail composition according to claim 1, wherein the acrylic resin is styrene/acrylates copolymer.

7. The nail composition according to claim 1, wherein the second resin is selected from the group consisting of a polyester, a polyurethane, acrylates copolymer, an epoxy resin, an alkyd resin, and a copolymer having trimellitic anhydride as a monomer.

8. The nail composition according to claim 7, wherein the copolymer having trimellitic anhydride as a monomer is adipic acid/neopentyl glycol/trimellitic anhydride copolymer.

9. The nail composition according to claim 1, wherein the second resin is selected from the group consisting of a polyester and an alkyd resin.

10. The nail composition according to claim 1, wherein the at least one plasticizer is present in an amount ranging from about 1% to about 15% by weight.

11. The nail composition according to claim 1, wherein the at least one plasticizer comprises dipropylene glycol dibenzoate, acetyl tributyl citrate, and sucrose acetate isobutyrate.

12. The nail composition according to claim 1, wherein the non-polar silicone copolymer is present in an amount ranging from about 0.01% to about 5% by weight.

13. The nail composition according to claim 1, wherein the non-polar silicone copolymer is acrylates/dimethicone copolymer, acrylates/stearyl acrylate/dimethicone acrylates copolymer, or acrylates/behenyl acrylate/dimethicone acrylates copolymer.

14. The nail composition according to claim 1, wherein the at least one solvent is present in an amount ranging from about 10% to about 70% by weight.

15. The nail composition according to claim 1, wherein the at least one solvent comprises ethyl acetate.

16. The nail composition according to claim 15, wherein the at least one solvent further comprises propyl acetate and isopropanol.

17. The nail composition according to claim 1, further comprising at least one pigment.

18. The nail composition according to claim 1, further comprising at least one pigment, the at least one pigment present in an amount greater than 0.5% by weight.

19. The nail composition according to claim 18, further comprising nitrocellulose.
